(19) United States
(12) Patent Application Publication
(10) Pub. No.: US 2025/0303081 A1
(43) Pub. Date: Oct. 2, 2025

(54) MEDICAL DEVICE

(71) Applicant: FUKUDA DENSHI CO., LTD., Tokyo (JP)

(72) Inventors: Takanori ONO, Tokyo (JP); Naoto SHIRAISHI, Tokyo (JP)

(21) Appl. No.: 19/092,328

(22) Filed: Mar. 27, 2025

(30) Foreign Application Priority Data
Mar. 29, 2024 (JP) 2024-056577

(51) Int. Cl. A61M 16/00

(52) U.S. Cl. CPC A61M 16/0066

(57) ABSTRACT

The medical device of the present disclosure includes the following: a first air blower that generates an airflow to be delivered to an airway of a patient; a flow path case in which a flow path for the airflow is formed; a circuit board provided above the flow path case; an AC/DC adapter provided below the flow path case; a second air blower provided below the flow path case and configured to cool the AC/DC adapter; a housing case that houses the first air blower, the flow path case, the AC/DC adapter, and the second air blower; and at least two openings that allow upper and lower spaces of the housing case separated by the flow path case to communicate with each other.

### CROSS REFERENCE TO RELATED APPLICATIONS

This application is entitled to and claims the benefit of Japanese Patent Application No. 2024-056577, filed on Mar. 29, 2024, the disclosure of which including the specification, drawings and abstract is incorporated herein by reference in its entirety.

### TECHNICAL FIELD

The technology of the present disclosure relates to medical devices, such as devices for treating sleep apnea syndrome.

### BACKGROUND ART

Sleep apnea syndrome (SAS) is a condition with a state where breathing stops (apnea) and a state where breathing becomes weak (hypopnea) repeating intermittently during sleep. Patients suffering from sleep apnea syndrome often do not get enough sleep, leading to daytime drowsiness, decreased concentration, an increased risk of serious accidents due to drowsiness while driving, and the like. Most patients suffering from sleep apnea syndrome exhibit symptoms of obstructive sleep apnea (OSA). Obstructive sleep apnea occurs when muscle tone decreases during inhalation in sleep, causing the upper airway to narrow.

For patients with such obstructive sleep apnea, continuous positive airway pressure (CPAP) therapy may be administered. CPAP therapy is a treatment method that prevents apnea during sleep by continuously supplying air to an airway of a patient to keep the airway open.

Traditionally, devices that perform CPAP therapy are referred to as sleep apnea syndrome treatment devices or CPAP devices. Herein, devices that perform CPAP therapy will be referred to as CPAP devices. CPAP devices are described in, for example, PTL 1 below.

A CPAP device includes an air blower, a flow sensor, and a controller, and is configured to generate an airflow suitable for expanding an airway of a patient. Some CPAP devices include a water tank to humidify the airflow to be sent to a patient, preventing the airway of the patient from drying out.

Such a CPAP apparatus is described in, for example, Japanese Patent Application Laid-Open No. 2023-071739.

In electronic devices using external power sources, power is input via an AC/DC adapter (which may also be referred to as an AC/DC converter or power adapter) which converts AC power (commercial power) to DC power. Traditionally, an AC/DC adapter is often provided outside a device and attached to a power cord.

However, having an AC/DC adapter attached to a device is obstructive and requires space. Especially in medical devices, where patients and healthcare professionals are often nearby, the AC/DC adapter is more likely to become an obstruction.

Therefore, incorporating the AC/DC adapter is possible, but an AC/DC converter generates a lot of heat, and thus requires heat dissipation measures. However, medical devices such as CPAP devices do not have a sufficient heat dissipation structure.

The present disclosure has been made in consideration of the above circumstances and provides a medical device capable of satisfactory heat dissipation.

### SUMMARY

One aspect of the medical device of the present disclosure includes: a first air blower that generates an airflow to be delivered to an airway of a patient;
a flow path case in which a flow path for the airflow is formed;
a circuit board provided above the flow path case;
an AC/DC adapter provided below the flow path case;
a second air blower provided below the flow path case and configured to cool the AC/DC adapter;
a housing case that houses the first air blower, the flow path case, the AC/DC adapter, and the second air blower; and
at least two openings that allow an upper space and a lower space of the housing case separated by the flow path case to communicate with each other.

### BRIEF DESCRIPTION OF THE DRAWINGS

FIG. 1 illustrates a CPAP device being attached to a patient;

FIG. 2A is a perspective view of the CPAP device from diagonally above;

FIG. 2B is a perspective view of the CPAP device from diagonally above;

FIG. 3 is an exploded perspective view of the CPAP device according to an embodiment;

FIG. 4 is a schematic diagram illustrating the path of an airflow;

FIG. 5 is a block diagram for explaining the configuration of the CPAP device according to the embodiment;

FIG. 6 is a schematic cross-sectional view illustrating the relationship between a housing case and a flow path case; and

FIG. 7 is a schematic perspective view for explaining a circulation path of a wind from a fan.

### DESCRIPTION OF EMBODIMENTS

Hereinafter, embodiments of the present disclosure will be described in detail with reference to the drawings.

### Configuration of CPAP Device According to Embodiment

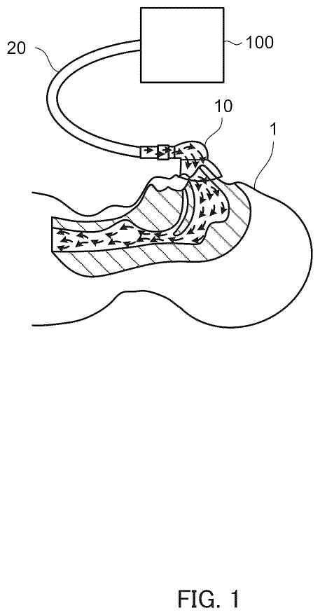

As illustrated in FIG. 1, CPAP device 100 is connected via tube 20 to mask 10 worn on the face of patient 1 suffering from sleep apnea syndrome, and delivers an airflow with a positive pressure to the upper airway of patient 1 to expand the upper airway. In the present embodiment, CPAP device 100 represents the main body of the CPAP device, and the CPAP device is configured to include CPAP device 100 as the CPAP device main body, mask 10, and tube 20.

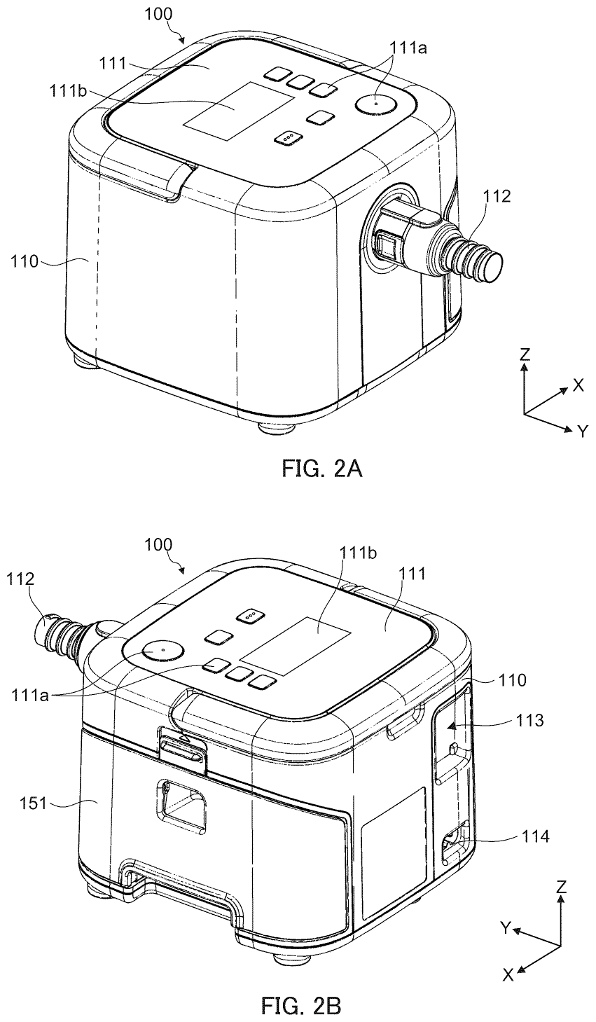

FIGS. 2A and 2B are perspective views of CPAP device 100 from diagonally above. Here, the +Z direction in the drawing indicates the upward direction of CPAP device 100, and the-Z direction indicates the downward direction of CPAP device 100. In addition, the +Y direction indicates the forward direction of CPAP device 100, and the-Y direction indicates the backward direction of CPAP device 100. Furthermore, the +X direction indicates the left direction of CPAP device 100, and the −X direction indicates the left direction of CPAP device 100.

As can be seen from FIG. 2A, tube connector 112, to which tube 20 (FIG. 1) is connected, protrudes from the front side surface of housing case 110 of CPAP device 100. In addition, operation panel 111 is provided on the upper part of housing case 110. Operation panel 111 is provided with operation input 111a including operation buttons, and display 111b.

As can be seen from FIG. 2B, intake port 113 and power connector 114 are provided on the rear side surface of housing case 110. Power connector 114 receives an AC power supply via a power cable. In addition, water tank 151 is detachably attached to the side surface of housing case 110.

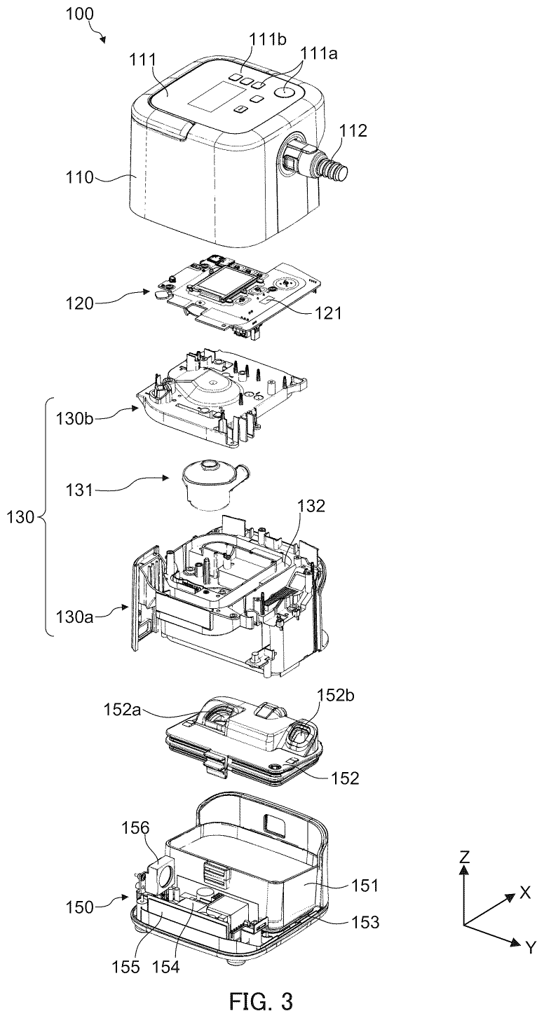

FIG. 3 is an exploded perspective view of CPAP device 100 according to the present embodiment.

CPAP device 100 mainly includes housing case 110, circuit board 120, flow path case 130, and base 150.

Housing case 110 has a rectangular cylindrical shape and houses circuit board 120, flow path case 130, and the like by being coupled from above to base 150.

Circuit board 120 is provided with a central processing unit (CPU), various driver circuits, and the like. In addition, in the present embodiment, circuit board 120 is provided with acceleration sensor 121.

Flow path case 130 is configured by fitting a lower case 130a and an upper case 130b together. Inside flow path case 130, blower 131 as the first air blower is disposed. Flow path 132, through which the wind generated by blower 131 passes, is formed inside flow path case 130.

A detachable water tank 151 is disposed on base 150. Air inlet 152a and air outlet 152b are formed on lid 152 of water tank 151. Air inlet 152a communicates with flow path 132 located inside flow path case 130. Air outlet 152b communicates with tube connector 112.

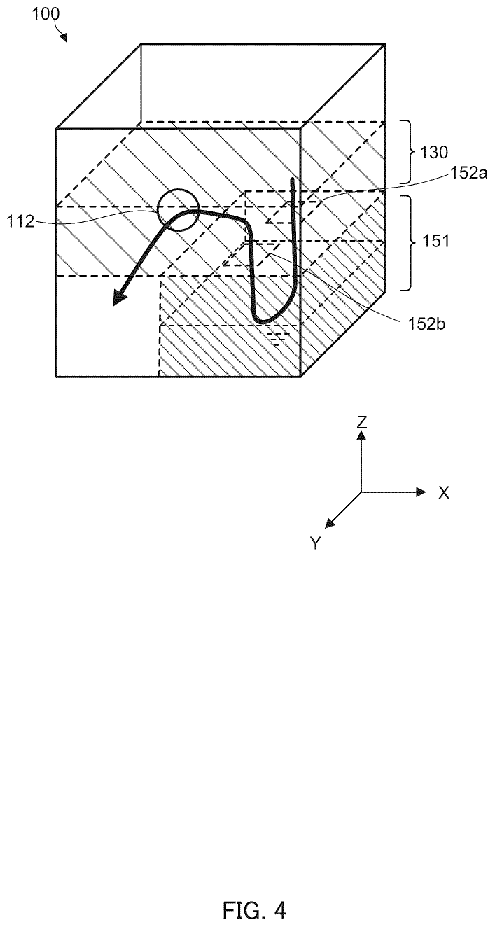

As can be seen from the schematic diagram in FIG. 4, an airflow (indicated by arrows in the drawing) generated by blower 131 thus passes through flow path 132 of flow path case 130 (FIG. 3), enters water tank 151 through air inlet 152a, is discharged from water tank 151 through air outlet 152b, and is supplied to the patient via tube connector 112.

Heater 153 is provided on the lower surface side of water tank 151. The water in water tank 151 is heated by heater 153 to create a high humidity state inside water tank 151. Therefore, the airflow to be supplied to the patient is humidified inside water tank 151. This reduces the drying of the airway of patient 1 by the airflow.

In addition, base 150 is provided with AC/DC adapter 154. AC/DC adapter 154 receives AC power from the outside through a power cord (not illustrated) connected to power connector 114 (FIG. 2B), converts the power to DC power, and supplies the DC power obtained by the conversion to circuit board 120 and the like.

A plurality of circuit components constituting AC/DC adapter 154 are covered from the bottom and both left and right sides by metal shield member 155 that has a U-shaped cross section taken along the XZ plane. Shield member 155 extends in the Y direction. Shield member 155 is a shield member made of a metal and surrounding AC/DC adapter 154, and at least both ends of the shield member in the longitudinal direction are open. Fan 156 is provided at the one open end side of shield member 155, as a second air blower to cool AC/DC adapter 154. Fan 156 is provided at a position facing AC/DC adapter 153.

As a result, AC/DC adapter 154 is efficiently cooled by a wind from fan 156, the wind flowing inside shield member 155 in the extending direction of shield member 155. In addition, electromagnetic noise generated from AC/DC adapter 154 is blocked by shield member 155.

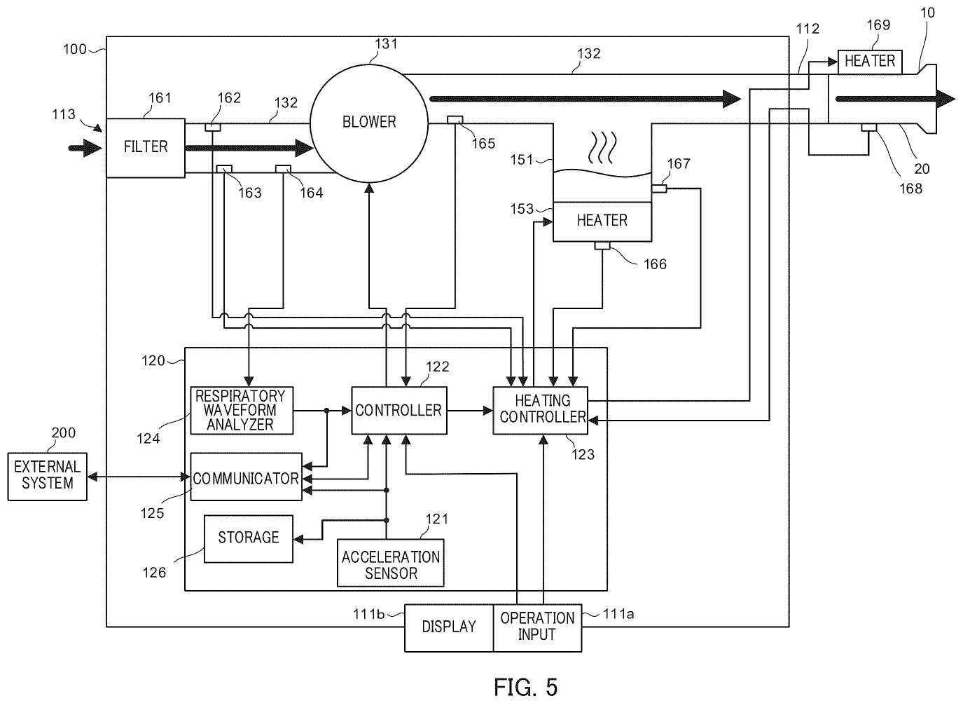

FIG. 5 is a block diagram for explaining the configuration of CPAP device 100. FIG. 5 mainly illustrates the configuration of a part that generates an airflow to be supplied to patient 1, thereby omitting AC/DC adapter 154, fan 156, and the like.

In addition to blower 131, filter 161, temperature sensor 162, humidity sensor 163, flow sensor 164, and pressure sensor 165 are provided at flow path 132 of CPAP device 100. In addition, temperature sensor 166 is attached to heater 153 that heats water tank 151, and a weight sensor 167 is attached to water tank 151

Circuit board 120 is provided with acceleration sensor 121, controller 122, heating controller 123, respiratory waveform analyzer 124, communicator 125, storage 126, and the like. In other words, circuit board 120 is equipped with circuit components to realize the functions of acceleration sensor 121, controller 122, heating controller 123, respiratory waveform analyzer 124, communicator 125, and storage 126. Specifically, circuit board 120 includes circuit patterns and components mounted on a flexible substrate, a rigid substrate, or a rigid-flexible substrate.

Controller 122, heating controller 123, and respiratory waveform analyzer 124 include a central processing unit (CPU), read only memory (ROM), random access memory (RAM), and the like. The CPU reads out a program corresponding to the processing content from the ROM and deploys the program in the RAM, and cooperates with the loaded program to realize the functions of controller 122, heating controller 123, and respiratory waveform analyzer 124. All or part of controller 122, heating controller 123, and respiratory waveform analyzer 124 may be formed from hardwired circuits such as an application specific integrated circuit (ASIC) or a field-programmable gate array (FPGA).

When blower 131 operates, external air enters flow path 132 through intake port 113 and filter 161. The temperature and humidity of the air in flow path 132 are measured by temperature sensor 162 and humidity sensor 163, and the measured temperature and humidity are sent to heating controller 123. Furthermore, heating controller 123 receives the heating set value and humidification set value (e.g., target temperature and target humidity) from operation input 111a, along with the temperature information of heater 153 from temperature sensor 166.

Heating controller 123 controls heater 153 based on the temperature and humidity information measured by temperature sensor 162 and humidity sensor 163, the heating set value and humidification set value from operation input 111a set by the user, and the temperature information of heater 153 from temperature sensor 166. For example, heating controller 123 controls heater 153 so that the temperature and humidity of the airflow to be supplied to patient 1 approach the heating set value and humidification set value.

In addition, heating controller 123 receives temperature information from temperature sensor 168 provided at tube 20. Heating controller 123 controls heater 169 provided at tube 20 based on this temperature information to prevent condensation within tube 20.

Flow sensor 164 is a differential pressure sensor that measures the respiratory flow of patient 1 and sends the measurement result to respiratory waveform analyzer 124. Respiratory waveform analyzer 124 performs analyze to acquire the respiratory waveform of patient 1 based on the respiratory flow and sends the acquired respiratory waveform as respiratory information to controller 122 and communicator 125.

The information on the pressure in flow path 132 measured by pressure sensor 165 is sent to controller 122. In addition, controller 122 receives pressure setting information (e.g., target pressure) from operation input 111a. Controller 122 controls the rotation of blower 131 based on the pressure information measured by pressure sensor 165, the respiratory information from respiratory waveform analyzer 124, and the pressure setting information from operation input 111a set by the user, thereby controlling the pressure of the airflow supplied to patient 1.

The acceleration information (detected value) measured by acceleration sensor 121 is sent to controller 122, communicator 125, and storage 126.

Controller 122 controls the operation of CPAP device 100 based on the acceleration information. Controller 122 performs determinations regarding CPAP device 100, such as drop, fall, impact, and vibration, based on the acceleration information, and when controller 122 determines that CPAP device 100 has dropped, fallen, been impacted, or vibrated, controller 122 stops the operation of all or part of CPAP device 100.

Communicator 125 communicates with external system 200. For example, the respiratory information obtained by respiratory waveform analyzer 124 is transmitted to external system 200 via communicator 125. This allows medical personnel at a distance from CPAP device 100 to know that patient 1 is experiencing apnea.

In addition, the acceleration obtained by acceleration sensor 121, the determination results regarding the drop, fall, impact, and vibration obtained by controller 122, and the operation stop information performed by controller 122 are transmitted to external system 200 via communicator 125. This allows the acceleration that occurred in CPAP device 100, drop, fall, impact, vibration, and operation stop to be known at external system 200. As a result, when external system 200 is a system server of a management company, such information can be utilized for maintenance work of CPAP device 100.

### Heat Dissipation Structure

Next, the heat dissipation structure in the present embodiment will be described in detail.

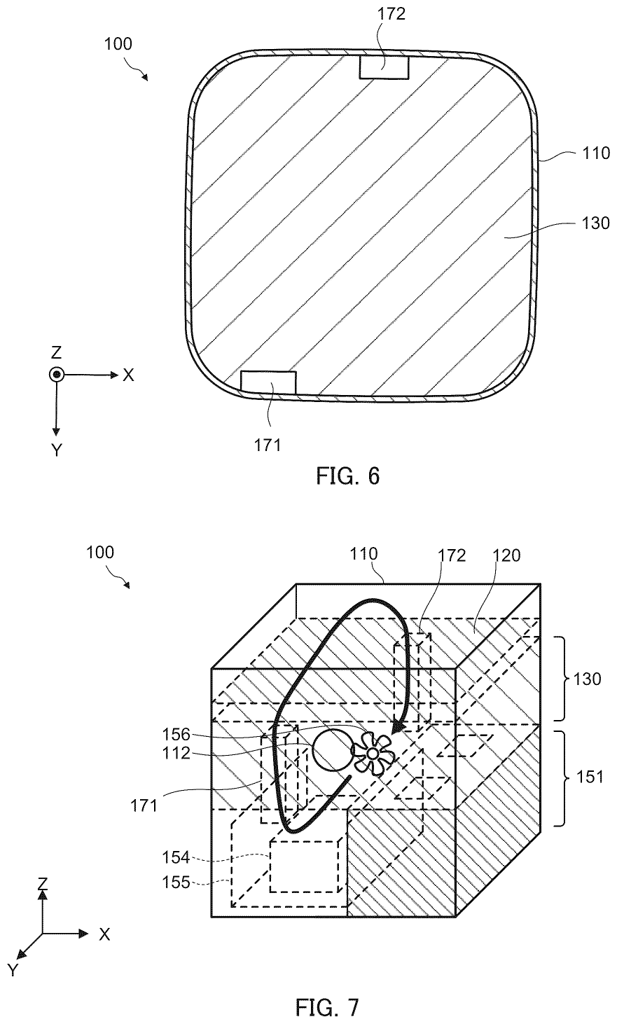

FIG. 6 is a schematic cross-sectional view illustrating the relationship between housing case 110 and flow path case 130. FIG. 6 is a schematic cross-sectional view taken along the XY plane at the position including housing case 110 in CPAP device 100.

As can be seen from FIG. 6, flow path case 130 is provided inside housing case 110 in such a way that the outer surface of flow path case 130 is in close contact with the inner surface of housing case 110 over almost the entire circumference. On the other hand, two recesses that are not in close contact with the inner surface of housing case 110 are formed in flow path case 130, and these two recesses respectively form first opening 171 and second opening 172 that extend in the vertical direction.

In other words, first and second openings 171 and 172 allow the upper and lower spaces of housing case 110 separated by flow path case 130 to communicate with each other. In the example of the present embodiment, first and second openings are respectively formed in the vicinities of the two side surfaces of housing case 110 that face each other with circuit board 120 (FIG. 7) in between (the front side surface and rear side surface in the present embodiment).

FIG. 7 is a schematic perspective view for explaining a circulation path of a wind from fan 156. An arrow in the drawing indicates the flow of the cooling wind.

The bottom surface and the left and right side surfaces of AC/DC adapter 154 are surrounded by metal shield member 155. As a result, electromagnetic noise generated from AC/DC adapter 154 is blocked by shield member 155.

Fan 156 is disposed at the one end side of shield member 155 in the longitudinal direction thereof so as to face AC/DC adapter 154. In addition, first opening 171 is formed at a position corresponding to the other end side of shield member 155. In other words, first opening 171 is formed downstream of AC/DC adapter 154 with respect to the wind generated by fan 156.

As a result, the wind from fan 156 flows along the U-shaped groove surrounded by shield member 155 to cool AC/DC adapter 154. The wind heated by cooling AC/DC adapter 154 is guided to first opening 171 by shield member 155 and further rises inside first opening 171. At this time, the wind passing through first opening 171 flows along housing case 110, and the heat of the wind is dissipated to the outside.

Next, the circulating wind from which the heat has been dissipated flows along circuit board 120. At this time, when there is a heat-generating circuit on circuit board 120, the circuit is cooled by the circulating wind.

The circulating wind then descends inside second opening 172. At this time, the wind passing through second opening 172 flows along housing case 110, and thus heat of the wind is dissipated to the outside. The circulating wind from which the heat has been dissipated is then drawn into fan 156.

In this way, the circulating wind heated by cooling AC/DC adapter 154 is cooled by first and second openings 171 and 172, thereby preventing a significant temperature rise inside CPAP device 100. In addition, the heat from AC/DC adapter 154 is diffused inside CPAP device 100 by the circulating wind, and thus local temperature rise in the vicinity of AC/DC adapter 154 is prevented.

Furthermore, the wind heated by cooling AC/DC adapter 154 is caused to directly hit the inner surface of housing case 110 but not directly hit flow path case 130, it is possible to prevent the airflow in flow path case 130 from being excessively heated. In a CPAP device, it is necessary to control the temperature of an airflow to be supplied to patient 1 within a desired temperature range. According to the heat dissipation structure of the present embodiment, even when hot air is generated by cooling AC/DC adapter 154, it is possible to prevent the temperature of the airflow from exceeding the desired temperature due to this hot air.

### SUMMARY

As described above, CPAP device 100 of the embodiment includes the following: a first air blower (blower 131) that generates an airflow to be delivered to the airway of patient 1; flow path case 130 in which a flow path for the airflow is formed; circuit board 120 provided above flow path case 130; AC/DC adapter 154 provided below flow path case 130; a second air blower (fan 156) provided below flow path case 130 and configured to cool AC/DC adapter 154; housing case 110 that houses first air blower (blower 131), flow path case 130, AC/DC adapter 154, and second air blower (fan 156); and at least two openings 171 and 172 that allow the upper and lower spaces of housing case 110 separated by flow path case 130 to communicate with each other.

As a result, CPAP device 100 that can perform satisfactory heat dissipation even when AC/DC adapter 154 is built in is realized.

In this manner, according to CPAP device 100 of the present embodiment, AC/DC adapter 154 can be built in with heat dissipation measures taken, and therefore a CPAP device with a neat exterior can be realized.

According to CPAP device 100 of the present embodiment, the heat in the device can be efficiently dissipated to the outside, and therefore, in the case of forming exhaust holes in the housing case, for example, the size of the exhaust holes can be reduced, or the temperature inside the housing case can be lowered to a predetermined value or less even without exhaust holes. Therefore, it is possible to reduce the leakage of mechanical noise to the outside and further enhance waterproofness and electrical safety.

The embodiments described above are merely examples of specific implementations of the present invention, and should not be construed as limiting the technical scope of the present invention. That is, the present invention can be implemented in various forms without departing from the spirit or main features thereof.

In addition to the above embodiment, a temperature sensor may be provided, for example, in the vicinity of the AC/DC adapter 154 or on the circuit board 120, and the rotation speed and on/off of fan 156 may be controlled based on the temperature obtained by the temperature sensor.

In addition, when a heat-generating components is mounted on the circuit board 120 at a position corresponding to the path of the circulating wind from fan 156, the heat of the component can be efficiently diffused, allowing the components to be cooled.

Furthermore, in the above embodiment, two openings 171 and 172 were formed as openings that allow the upper and lower spaces of housing case 110 separated by flow path case 130 to communicate with each other, but three or more openings may be formed to allow the upper and lower spaces of housing case 110 separated by flow path case 130 to communicate with each other.

The above embodiment describes a case where openings 171 and 172 are formed by creating recesses in flow path case 130, but the position and shape of the openings and members for forming the openings are not limited to this case. For example, openings may be formed by creating holes or grooves in housing case 110. Openings may also be formed by creating holes or grooves in flow path case 130. When forming holes or grooves in flow path case 130, it is preferable to form them along housing case 110 in the vicinity of housing case 110, in view of heat dissipation efficiency. Moreover, the openings do not necessarily have to be in the shape of a straight line extending vertically, but may be a straight line extending diagonally or have a bent shape. In short, the opening may be any opening that allows the upper and lower spaces of housing case 110 to communicate with each other.

Furthermore, the technology of the present disclosure is not limited to CPAP devices, but can also be applied to other medical devices such as adaptive servo ventilation (ASV) devices and nasal high flow (NHF) devices and other respiratory support devices.
(1) One aspect of the medical device of the present disclosure includes the following: a first air blower that generates an airflow to be delivered to an airway of a patient; a flow path case in which a flow path for the airflow is formed; a circuit board provided above the flow path case; an AC/DC adapter provided below the flow path case; a second air blower provided below the flow path case and configured to cool the AC/DC adapter; a housing case that houses the first air blower, the flow path case, the AC/DC adapter, and the second air blower; and at least two openings that that allow the upper and lower spaces of the housing case to communicate with each other—the spaces separated by the flow path case.

As a result, circulation occurs between the lower and upper spaces of the flow path case through at least two openings, and therefore, medical devices with satisfactory heat dissipation are realized.
(2) In one aspect of the medical device of the present disclosure in the above (1), the at least two openings are formed in or along the housing case.

As a result, heat inside the housing case is more likely to dissipate to the outside of the housing case, thereby improving heat dissipation efficiency.
(3) In one aspect of the medical device of the present disclosure in the above (1) or (2), the second air blower is disposed at a position facing the AC/DC adapter, and a first opening of the at least two openings is formed downstream of the AC/DC adapter with respect to a wind generated by the second air blower.

As a result, the heat from the AC/DC adapter, which is the main heat source, can be circulated efficiently, and therefore the temperature rise inside the housing case can be effectively prevented.
(4) In one aspect of the medical device of the present disclosure in the above (1), a first opening and a second opening of the at least two openings are respectively formed in the vicinities of two side surfaces of the housing case—the two side surfaces facing each other with the circuit board in between.

As a result, the circulating air flows through the openings and passes through the circuit board, and therefore the heat of the circuit board can also be efficiently dissipated.
(5) In one aspect of the medical device of the present disclosure in the above (1), a wind having passed through the AC/DC adapter passes through the flow path case from the lower side to the upper side of the flow path case via a first opening of the at least two openings, and the wind having passed through the first opening and the circuit board passes through the flow path case from the upper side to the lower side via a second opening of the at least two openings—the wind is from the second air blower. Therefore, the wind from the second air blower is circulated between the lower and upper spaces of the flow path case via the first and second openings.
(6) One aspect of the medical device of the present disclosure, in the above (1) or (2), further includes a shield member made of a metal and surrounding the AC/DC adapter, and at least both ends of the shield member in the longitudinal direction of the shield member are open. In the medical device, the second air blower is disposed at the one end side of the shield member in the longitudinal direction, and a first opening of the two openings is formed at a position corresponding to the other end side, the one end and the other end being open.

As a result, electromagnetic noise generated from the AC/DC adapter 154 is blocked by the shield member, and the heat generated by the AC/DC adapter is guided to the first opening by the shield member.

### REFERENCE SIGNS LIST

1 Patient
10 Mask
20 Tube
100 CPAP Device
110 Housing Case
111 Operation panel
112 Tube Connector
113 Intake port
114 Power Connector
120 Circuit Board
121 Acceleration sensor
130 Flow path case
131 Blower
132 Flow path
150 Base
151 Water tank
152 Lid
152a Air inlet
152b Air outlet
153, 169 Heater
154 AC/AD adapter
155 Shield member
156 Fan
122 Controller
123 Heating controller
125 Communicator
126 Storage
171 First opening
172 Second opening

1. A medical device comprising:
a first air blower that generates an airflow to be delivered to an airway of a patient;
a flow path case in which a flow path for the airflow is formed;
a circuit board provided above the flow path case;
an AC/DC adapter provided below the flow path case;
a second air blower provided below the flow path case and configured to cool the AC/DC adapter;
a housing case that houses the first air blower, the flow path case, the AC/DC adapter, and the second air blower; and
at least two openings that allow an upper space and a lower space of the housing case separated by the flow path case to communicate with each other.

2. The medical device according to claim 1, wherein the at least two openings are formed in or along the housing case.

3. The medical device according to claim 1, wherein:
the second air blower is disposed at a position facing the AC/DC adapter, and
a first opening of the at least two openings is formed downstream of the AC/DC adapter with respect to a wind generated by the second air blower.

4. The medical device according to claim 1, wherein
a first opening and a second opening of the at least two openings are respectively formed in vicinities of two side surfaces of the housing case, the two side surfaces facing each other with the circuit board in between.

5. The medical device according to claim 1, wherein
a wind having passed through the AC/DC adapter passes through the flow path case from a lower side to a upper side of the flow path case via a first opening of the at least two openings, and the wind having passed through the first opening and the circuit board passes through the flow path case from the upper side to the lower side via a second opening of the at least two openings, so that the wind circulates between the lower space and the upper space of the flow path case via the first opening and the second opening, the wind being from the second air blower.

6. The medical device according to claim 1, further comprising:
a shield member made of a metal and surrounding the AC/DC adapter, the shield member being a shield member in which at least both ends in a longitudinal direction of the shield member are open, wherein
the second air blower is disposed at a side of one end of the shield member in the longitudinal direction, and a first opening of the at least two openings is formed at a position corresponding to a side of another end of the shield member, the one end and the other end both being open.
